(19) United States
(12) Patent Application Publication
(10) Pub. No.: US 2014/0079729 A1
(43) Pub. Date: Mar. 20, 2014

(54) METHOD FOR IMPROVING ENDOTHELIAL FUNCTION AND DECREASING CARDIOVASCULAR MORBIDITY USING SHILAJIT

(71) Applicant: Natreon, Inc., New Brunswick, NJ (US)

(72) Inventor: Sanyasi R. Kalidindi, East Brunswick, NJ (US)

(73) Assignee: Natreon, Inc., New Brunswick, NJ (US)

(21) Appl. No.: 14/026,711

(22) Filed: Sep. 13, 2013

Related U.S. Application Data
(60) Provisional application No. 61/701,399, filed on Sep. 14, 2012.

(51) Int. Cl. A61K 38/17; A61K 31/366; A61K 35/10

(52) U.S. Cl. CPC A61K 38/1709; A61K 35/10; A61K 31/366
USPC 424/195.18

(57) ABSTRACT

Shilajit in a standardized composition produces a significant improvement in several cardiovascular parameters including RI, AIx and SEVR. Further, significant reductions in malondialdehyde and increases in nitric oxide levels are provided suggesting improvement in endothelial function. Shilajit may be used to reduce inflammatory biomarker HsCRP levels significantly compared to baseline and placebo. Additionally, Shilajit can provide significant improvement in lipid parameters including total cholesterol, LDL-C, and HbA1c (%) Inhibition of platelet aggregation using Shilajit performed using ADP as aggregant also provides highly significant inhibition of platelet aggregation compared to baseline and with placebo. Thus, Shilajit may be used for improvement of endothelial function and to help reduce cardiovascular morbidity, particularly for the diabetic individual.

This application claims the benefit of earlier filed U.S. Provisional Application No. 61/701,399, filed on Sep. 14, 2012, which is hereby incorporated by reference herein.

### FIELD OF THE INVENTION

The present invention relates to improvement of human endothelial function and cardiovascular parameters through use of the herbo-mineral Shilajit.

### BACKGROUND

Cardiovascular disease (CVD) is the number one cause of death globally. Smoking, hypertension, high LDL cholesterol, low HDL cholesterol and diabetes mellitus (DM) are the five major risk factors for CVD. Diabetes is associated with an increased risk of atherosclerosis, which may result in coronary artery disease (CAD) (A. Pandolfi, et al., “Chronic hyperglycemia and nitric oxide bioavailability play a pivotal role in proatherogenic vascular modifications,” Genes & Nutrition (2007) 2 (2): 195-208). Physiological impairments that link DM with a marked increase in atherosclerotic vascular disease include platelet hyper-reactivity, a tendency for negative arterial remodeling, impaired fibrinolysis, increased inflammation, and endothelial dysfunction.

Endothelial dysfunction, present at disease onset, may be the cause of atherogenesis that is present throughout the course of DM and associated with late-stage adverse outcomes (Panwar, et al., “Atherothrombotic risk factors & premature coronary heart disease in India: A case-control study,” Indian J. Med. Res. (July 2011) 134: 26-32). The endothelial dysfunction results from reduced bioavailability of the vasodilator nitric oxide (NO) mainly due to accelerated NO degradation by reactive oxygen species (J. A. Beckman, “Pathophysiology of Vascular Dysfunction in Diabetes,” Cardiology Rounds (December 2004) Volume 8, Issue 10). A currently favored hypothesis is that oxidative stress, through a single unifying mechanism of superoxide production, is the common pathogenic factor leading to insulin resistance, β-cell dysfunction, impaired glucose tolerance (IGT) and ultimately to Type 2 DM (T2DM). Furthermore, this mechanism has been implicated as the underlying cause of both the macrovascular and microvascular complications associated with Type 2 DM. It follows that therapies aimed at reducing oxidative stress would benefit both patients with T2DM and those at risk for developing diabetes (Potneza, et al., “Endothelial Dysfunction in Diabetes: From Mechanism to Therapeutic Targets,” Current Medicinal Chemistry (2009) 16: 94-112; S. E. Inzucchi, “Oral Antihyperglycemic Therapy for Type 2 Diabetes. Scientific Review and Clinical Applications,” Journal of American Medical Association (Jan. 16, 2002-Vol 287, No. 3, pp. 360-372; and Wright, et al., “Oxidative stress in type 2 diabetes: the role of fasting and postprandial glycaemia,” Int. J. Clin. Pract. (2006 March) 60(3): 308-314).

Many natural products possess potent antioxidant, anti-inflammatory and cardio-protective properties and are used by patients with increased risk of cardiovascular morbidity and mortality in order to treat or prevent disease and/or reduce symptoms.

Among them, Shilajit is an herbo-mineral drug, which oozes out from a special type of mountain rocks in the peak summer months. It is found at high altitudes ranging from 1000-5000 meters. The active constituents of Shilajit contain dibenzo-alpha-pyrones and related metabolites, small peptides (constituting non-protein amino acids), some lipids, and carrier molecules (fulvic acids). See, Ghosal, S., et al., “Shilajit Part 1—Chemical constituents,” J. Pharm. Sci. (1976) 65:772-3; Ghosal, S., et al., “Shilajit Part 7—Chemistry of Shilajit, an immunomodulatory ayurvedic rasayana,” Pure Appl. Chem. (IUPAC) (1990) 62:1285-8; Ghosal, S., et al., “The core structure of Shilajit humus,” Soil Biol. Biochem. (1992) 23:673-80; and U.S. Pat. Nos. 6,440,436 and 6,869,612 (and references cited therein); all hereby incorporated by reference herein.

Shilajit (PrimaVie®) finds extensive use in Ayurveda, for diverse clinical conditions. For centuries people living in the isolated villages in Himalaya and adjoining regions have used Shilajit alone, or in combination with, other plant remedies to prevent and combat problems with diabetes (Tiwari, V. P., et al., “An interpretation of Ayurvedica findings on Shilajit,” J. Res. Indigenous Med. (1973) 8:57). Moreover being an antioxidant it will prevent damage to the pancreatic islet cell induced by the cytotoxic oxygen radicals (Bhattacharya S. K., “Shilajit attenuates streptozotocin induced diabetes mellitus and decrease in pancreatic islet superoxide dismutase activity in rats,” Phytother. Res. (1995) 9:41-4; Bhattacharya S. K., “Effects of Shilajit on biogenic free radicals,” Phytother. Res. (1995) 9:56-9; and Ghosal, S., et al., “Interaction of Shilajit with biogenic free radicals,” Indian J. Chem. (1995) 34B:596-602). It has been proposed that the derangement of glucose, fat and protein metabolism during diabetes, results into the development of hyperlipidemia. In one study, Shilajit produced significant beneficial effects in lipid profile in rats (Trivedi N. A., et al., “Effect of Shilajit on blood glucose and lipid profile in alloxan-induced diabetic rats,” Indian J. Pharmacol. (2004) 36(6):373-376). However, some drugs elicit a response in animals but may not do so in humans. Thus, the present invention relates to evaluating the effect of Shilajit on endothelial function and cardiovascular morbidity in humans.

In view of the above, it would be desirable to provide a method of using Shilajit for improvement of endothelial function and other cardiovascular parameters, and to help reduce cardiovascular morbidity in a human patient.

### SUMMARY OF THE INVENTION

An objective of the present invention is to develop a method of using Shilajit compositions for improving endothelial function and cardiovascular health in patients with Type 2 diabetes mellitus as well as in healthy subjects.

In one embodiment, a method of treating or preventing endothelial dysfunction is provided including administering to an individual in need thereof an effective amount of a composition comprising Shilajit and a pharmaceutically acceptable carrier, wherein endothelial function is improved.

In another embodiment, a method of treating a diabetic individual suffering from type 2 diabetes mellitus is provided including administering to an individual in need thereof an effective amount of a composition comprising Shilajit and a pharmaceutically acceptable carrier, wherein endothelial function is improved.

In yet another embodiment, a method of treating a diabetic individual suffering from type 2 diabetes mellitus is provided including administering to an individual in need thereof an effective amount of a composition comprising Shilajit and a pharmaceutically acceptable carrier, wherein a blood lipid parameter is improved.

### DETAILED DESCRIPTION

In one aspect, the present invention reveals the usefulness of Shilajit compositions in improving endothelial function and cardiovascular health in patients with Type 2 diabetes mellitus as well as in healthy subjects.

Patients with diabetes have vascular complications and endothelial dysfunction is one of the early prognostic markers of atherosclerosis which may eventually result in cardiovascular disease. Studies have reported that endothelial dysfunction occurs in patients with diabetes much earlier than clinical manifestations of diabetic vascular complications (Schalkwijk, et al., “Vascular complications in diabetes mellitus: the role of endothelial dysfunction,” Clinical Science (2005) 109: 143-159). Diabetes is associated with accelerated atherosclerosis and microvascular complications which may be major causes of morbidity and mortality, as discussed above. Endothelial cell dysfunction is emerging as a key component in the pathophysiology of cardiovascular abnormalities associated with diabetes mellitus.

Increased arterial stiffness, as measured by pulse wave analysis, is associated with cardiovascular risk factors and established coronary artery disease. Pulse wave analysis is simple and reproducible to stratify cardiac risk in diabetes. Whilst arterial compliance is determined predominantly by structural factors, the vascular endothelium is also involved. The vascular endothelium contributes to vascular tone and endothelial dysfunction is implicated as an early functional alteration predating structural changes of the vasculature. Conventional cardiac risk factors such as dyslipidemia, hypertension, smoking, and Type 2 diabetes are associated with impaired endothelial function. The intact endothelium promotes vasodilatation principally via the release of NO—originally also called endothelium derived relaxing factor. Endothelium dependent vasodilators reduce pulse wave velocity suggesting nitric oxide (NO) plays a role in determining arterial distendability. Free radical NO has emerged as a fundamental signaling device regulating virtually every critical cellular function and is a potent mediator of cellular damage in many conditions. Nitric oxide is produced in endothelial cells from the substrate L-Arginine via endothelial Nitric oxide synthatase (eNOS). Elevated asymmetric dimethylarginine levels cause coupling, a mechanism which leads to decreased NO bioavailability. The endothelial dysfunction associated with diabetes has been attributed to lack of bioavailable nitric oxide due to reduced ability to synthesize NO from L-Arginine. New basic research insights provide possible mechanisms underlying the impaired NO bioavailability in Type 2 diabetes.

Use of herbs and/or herbo-minerals for the treatment of cardiovascular diseases and diabetes in Ayurveda, Chinese and Unani systems of medicine has provided new leads to understanding the pathophysiology of these diseases. Therefore, it is rational to use our natural resources for identifying and selecting inexpensive and safer approaches for the management of cardiovascular disease along with current therapy.

As discussed above, Shilajit may be a useful component for therapeutic treatment of vascular conditions and for palliative treatment of endothelial dysfunction.

Study in Diabetic Subjects

A prospective, randomized, double blind clinical study was conducted with twenty-five diabetic patients enrolled in the study. Patients included in the study were of either sex, aged 18-75 years, fasting plasma glucose of ≧110 mg/dL, a glycosylated haemoglobin (HbA1c) between 7% and 9% and taking a stable dose of anti-diabetic treatment (Metformin 1500-2500 mg/day) for the past 8 weeks prior to the screening visit; and having endothelial dysfunction defined as ≦6% change in reflection index (RI) on post salbutamol challenge test. Patients with severe uncontrolled hyperglyceamia, uncontrolled hypertension, cardiac arrhythmia, impaired hepatic or renal function, history of malignancy or stroke, smoking, chronic alcoholism, or any other serious disease requiring active treatment and treatment with any other herbal supplements, were excluded from the study.

Study design.

After screening, all the eligible subjects were randomized to receive either one of the two treatments for a duration of 12 weeks: Group 1 received one capsule containing 250 mg Shilajit (PRIMAVIE® 250 mg capsules) twice daily orally, and Group 2 received one capsule of Placebo twice daily orally. Subjects were asked to report for follow up visits at 4, 8, and 12 weeks of therapy. At each visit, they were evaluated for efficacy and safety. Pharmacodynamic evaluation for endothelial function was conducted at every visit. Blood samples were collected for evaluation of biomarkers before and at end of the treatment. Inhibition of platelet aggregation was also studied with the two treatments. Safety lab investigations for hematological, hepatic and renal biochemical parameters were conducted before and at the end of the study, and also as and when required (in case of any adverse drug reaction (ADR)). Subjects were interviewed for the presence of ADRs and the same was recorded in the case report form. Compliance to therapy was assessed by pill count method.

The active ingredients used in the capsules may have the following compositions.

Shilajit (PrimaVie®, available from Natreon, Inc., New Brunswick, N.J.) is a standardized dietary supplement ingredient extracted and processed from Shilajit bearing rocks, containing not less than about 50% to 60% by weight fulvic acids (FAs), at least about 10% by weight dibenzo-α-pyrone chromoproteins, and at least 0.3%, or more, by weight total dibenzo-α-pyrones (DBPs). Water content is about 6%, or less, by weight. Water-soluble extractive value is about 80% (w/w), or greater.

Procedure for Assessment of Endothelial Function.

A salbutamol (albuterol) challenge test employing digital volume plethysmography was used to assess endothelial function as reported by Chowienczyk et al., “Photoplethysmographic assessment of pulse wave reflection: blunted response to endothelium dependant beta 2-adrenergic vasodilation in type 2 diabetes mellitus,” J. Am. Coll. Cardiol. (1999 Dec) 34(7):2007-14; and Naidu, et al., “Comparison of two β₂ adrenoceptor agonists by different routes of administration to assess human endothelial function,” Indian J. Pharmacol. (2007) 39:168-9. The patients were examined in supine position after 5 minutes of rest. A digital volume pulse (DVP) was obtained using a photo plethysmograph (Pulse Trace PCA2, PT200, Micro Medical, Gallingham, Kent, UK) transmitting infrared light at 940 nm, placed on the index finger of the right hand. The signal from the plethysmograph was digitized using a 12 bit analogue to digital converter with a sampling frequency of 100 Hz. DVP waveforms were recorded over 20 second period and the height of the late systolic/early diastolic portion of the DVP was expressed as a percentage of the amplitude of the DVP to yield the reflection index (RI), per the procedure described in detail by Millasseau et al., “Determination of age related increases in large artery stiffness by digital pulse contour analysis,” Clinical Science (2002) 103: 371-377. After DVP recordings had been taken, three measurements of reflection index (RI) were calculated and the mean value was determined. Patients were then administered 400 μg of salbutamol by inhalation. After 15 minutes three measurements of RI were obtained again and the difference in mean RI before and after administration of salbutamol was used for assessing endothelial function. A change of ≦6% in RI post salbutamol was considered as endothelial dysfunction.

Measurement of Wave Reflection Indices

Augmentation index (AIx) and augmented pressure of the central (aortic) pressure waveform were measured as indices of wave reflections. Augmented pressure is the pressure added to the incident wave by the returning reflected one and represents the pressure boost that is caused by wave reflection and with which the left ventricle must cope.

Augmentation pressure (AP) is the contribution that wave reflection makes to systolic arterial pressure, and it is obtained by measuring the reflected wave coming from the periphery to the centre. Reduced compliance of the elastic arteries causes an earlier return of the ‘reflected wave’, which arrives in systole rather than in diastole, causing a disproportionate rise in systolic pressure and an increase in pulse pressure (PP), with a consequent increase in left ventricular afterload and impaired coronary perfusion.

The augmentation index (AIx) is an indirect measure of arterial stiffness and increases with age, and it is calculated as AP (augmentation pressure) divided by PP×100 to give a percentage. With an increase in stiffness there is a faster propagation of the forward pulse wave as well as a more rapid reflected wave. AP and AIx both increase with age. Augmentation index is commonly accepted as a measure of the enhancement (augmentation) of central aortic pressure by a reflected pulse wave.

Augmentation index is calculated from pulse waves of the common carotid artery recorded by applanation tonometry (SphygmoCor; AtCor Medical, Sydney, Australia). The systolic part of central arterial waveform is characterized by two pressure peaks. The first peak is caused by left ventricular ejection, whereas the second peak is a result of wave reflection. The difference between both pressure peaks reflects the degree to which central arterial pressure is augmented by wave reflection. Augmentation index (%) is defined as the percentage of the central pulse pressure which is attributed to the reflected pulse wave and, therefore, reflects the degree to which central arterial pressure is augmented by wave reflection.

Augmentation index is a sensitive marker of arterial status, in that:

Augmentation index has been shown to be a predictor of adverse cardiovascular events in a variety of patient populations, and higher augmentation index is associated with target organ damage, and

Augmentation index can distinguish between the effects of different vasoactive medications when upper arm blood pressure and pulse wave velocity do not.

The augmentation index is thus a composite measure of the magnitude of wave reflections and arterial stiffness, which affects timing of wave reflections. Because the augmentation index is influenced by changes in heart rate (HR), it was also accordingly corrected (AIx@75). The augmentation index was measured by using a validated, commercially available system (SphygmoCor; AtCor Medical, Australia) that employs the principle of applanation tonometry and appropriate acquisition and analysis software for noninvasive recording and analysis of the arterial pulse. In brief, from radial artery recordings, the central (aortic) arterial pressure was derived with the use of a generalized transfer function that has been shown to give an accurate estimate of the central arterial pressure waveform and its characteristics.

The subendocardial viability index, an indicator of myocardial workload and perfusion (O₂ supply vs. demand) was calculated as the ratio of the integral of diastolic pressure and time to the integral of systolic pressure and time. Low SEVR (Subendocardial viability ratio) has been shown to be associated with coronary artery disease, decreased coronary flow reserve in patients with healthy coronary arteries, severity of type I and type II diabetes, decreased renal function, and a history of smoking

Assessment of Arterial Stiffness (baPWV, ABI)

Brachial-ankle pulse wave velocity (baPWV) is also used to evaluate arterial stiffness. Pulse wave velocity is the speed at which the blood pressure pulse travels from the heart to the peripheral artery after blood rushes out during contraction. It is mainly used to evaluate stiffness of the artery wall. Pulse wave velocity increases with stiffness of the arteries. The PTT (Pulse Transit Time) of each segment is calculated from the waveform taken from each sensor. Pulse wave velocity is defined in Equation (1):

$$PWV = \frac{L ( distance )}{PTT ( Pulse Transit Time )} \qquad Equation ( 1 )$$

This method calculates heart-brachial PWV of both upper limbs, heart-ankle PWV of both lower limbs, brachial-ankle PWV of both right and left limb pairs, and effective estimated carotid-femoral PWV is calculated. See Equations (2), (3), and (4):

$$\begin{matrix}ha PWV ( heart ankle PWV ) - \frac{Lha}{PTTha} & Equation ( 2 ) \\ hb PWV ( heart brachial PWV ) - \frac{Lhb}{PTThb} & Equation ( 3 ) \\ ba PWV ( brachial ankle PWV ) - \frac{Lba}{PTTba} & Equation ( 4 )\end{matrix}$$

Where

Lha=Distance between heart and respective ankle

Lhb=Distance between heart and respective brachium.

Lba=Distance between respective brachium and ankle

Brachial Ankle Pulse Wave Velocity (baPWV), Ankle Brachial Index (ABI) and Blood Pressure (BP) were measured using an automatic waveform analyzer (model BP-203 RPE; Colin Medical Technology, Komaki, Japan). Measurements were taken with patients lying in a supine position after 5 minutes of rest in that position. Occlusion and monitoring cuffs were placed snugly around both sites of the upper and lower extremities of patients. Pressure waveforms of the brachial and tibial arteries were then recorded simultaneously by an oscillometric method. Measurement of right and left baPWV was obtained for an average of 10 seconds. The average of left and right baPWV will be used for analysis.

Method for Recording of Cardiac Output (Lt/Min)

Recording of cardiac output (CO) was performed using L&T Nivomon monitor (Larsen & Toubro Ltd., Mumbai, India). Noninvasive continuous cardiac output monitor with peripheral blood flow measurement option. This equipment is very useful and versatile. It calculates many cardiac parameters directly including cardiac output. It works on the features of impedance plethysmography principle and has tetrapolar configuration. One advantage is that this equipment directly calculates the cardiac output along with other parameters using the pulse wave.

Biomarker Evaluation

Nitric oxide, MDA, Glutathione and levels were estimated spectrophotometrically and HsCRP (high sensitivity C-reactive protein) by ELISA method. Malondialdehyde (MDA) levels were determined as described in Vidyasagar, et al., “Oxidative stress and antioxidant status in acute organophosphorous insecticide poisoning,” Indian J. Pharmacol. (April 2004) 36(2): 76-79. Glutathione (GSH) levels were determined as described in G. L. Ellman, Arch. Biochem. Biophys. (1959) 82: 70-77 (original determination). Nitric oxide levels were estimated spectrophotometrically as described in Miranda, et al., “A Rapid, Simple Spectrophotometric Method for Simultaneous Detection of Nitrate and Nitrite,” NITRIC OXIDE: Biology and Chemistry (2001) Vol. 5, No. 1, pp. 62-71.

Method For Evaluating Platelet Function

The effect of Shilajit (PrimaVie®) and Placebo on platelet function was determined by the following procedure. After assessing the eligibility of the subject by performing the evaluation of endothelial dysfunction, i.e., a change of ≦6% in RI post salbutamol, the platelet function test was carried in a dual channel platelet aggregometer instrument (Wheecon chronologue dual channel platelet aggregometer, Wheecon Instruments Pvt. Ltd., Chemai, Tamilnadu, India).

About 9 ml of blood sample was collected in a 10 ml plastic test tube containing 1 ml of 3.8% sodium citrate from the cubital vein of the subject at baseline and after post treatment in both the groups. The test was performed immediately within a time period of one and a half hour from collection. The samples were centrifuged at 800 rpm for 15 minutes to obtain a platelet rich plasma. The same sample was centrifuged at 2500 rpm for 10 minutes so as to get a poor platelet plasma sample. The aggregometer was switched about 30 minutes before the test to allow the heating block to warm up to 37° C. Then the test was performed in duplicate by taking 0.5 ml of platelet rich plasma using 5 μA of ADP (adenosine di-phosphate) (2 μm/ml) in cuvettes containing stir bars. The speed of the stir bars was adjusted to 1200 rpm so as to facilitate the aggregation of the platelets. The platelet-poor plasma sample was kept as a reference. The readings were recorded at baseline and after treatment with ADP. The percentage aggregation at baseline and the percentage inhibition of platelet aggregation on post treatment with the two treatments was calculated.

Safety Assessments

All the subjects had undergone complete physical examination, safety lab evaluations at baseline and at the end of the treatment. Samples were collected after an overnight fast of 12 hrs after the last dose of medication for determination of haemoglobin, HbA1c, blood urea and serum creatinine, liver function test, and lipid profile (Total cholesterol, High density lipoprotein cholesterol (HDL-C), low density lipoprotein cholesterol (LDL-C)). Plasma glucose, liver function test, blood urea, serum creatinine and HbA1c were measured using appropriate standard techniques.

Efficacy and Safety Parameters

The primary efficacy measure was a change in endothelial dysfunction as assessed by more than 6% change in reflection index at 12 weeks in all the treatment groups. Secondary efficacy measures include change in oxidative stress markers, serum levels of nitric oxide at 12 weeks in all the treatment groups and also evaluation of safety and tolerability of the test medications.

Data Analysis

Data are expressed as mean±SD (standard deviation). ANOVA and paired and unpaired t-test were performed for within group and between groups analysis respectively. A p-value <0.05 was considered to be statistically significant. All statistical analysis were performed using the Prism Graphpad 4 (GraphPad Software, Inc., La Jolla, Calif., USA).

Results of Study

Total of 25 subjects were screened and 20 eligible subjects completed the study. Ten subjects each in Shilajit (PrimaVie®) 250 mg and Placebo groups completed the study, as shown in Table 1.

TABLE 1
Demographic characteristics of the two study Groups

| Parameter | Shilajit (PrimaVie ®) | Placebo |
|---|---|---|
| Total No. | n = 10 | n = 10 |
| Gender (M/F) | 8/2 | 7/3 |
| Age (yrs) | 55.40 ± 10.71 | 56.90 ± 8.81 |
| Weight (Kg) | 65.50 ± 8.99 | 66.30 ± 6.46 |
| BMI (Kg/m²) | 24.73 ± 3.17 | 25.48 ± 2.10 |

The detailed demographic characteristics of the two study groups are shown above in Table 1. There was no significant difference between treatment groups in baseline characteristics including age, weight & body mass index (BMI).

TABLE 2
Effect of Shilajit (PrimaVie ®) & Placebo on pharmacodynamic cardiovascular
parameters after 12 weeks of treatment - All values expressed as Mean ± SD

| | Shilajit (PrimaVie ®) n = 10 | | Placebo n = 10 | |
|---|---|---|---|---|
| Parameter | Pretreatment | Post treatment | Pretreatment | Post treatment |
| RI (%) | −2.54 ± 1.72 | −8.61 ± 2.51 $ | −2.01 ± 0.71 | 0.07 ± 3.17 |
| AIx (%) | 145.8 ± 13.88 | 137.3 ± 9.82 # | 142.8 ± 15.32 | 143.5 ± 15.14 |
| SEVR (%) | 144.0 ± 27.90 | 154.3 ± 27.47 # | 146.6 ± 21.94 | 147.3 ± 21.20 |
| ABI | 1.05 ± 0.04 | 1.06 ± 0.05 NS | 1.04 ± 0.05 | 1.05 ± 0.06 |
| PWV (cm/s) | 1560 ± 203.4 | 1478 ± 128.8 NS | 1601 ± 141.1 | 1603 ± 146.2 |
| CO (Lt/min) | 5.09 ± 1.31 | 5.29 ± 1.12 NS | 4.44 ± 0.63 | 4.33 ± 0.58 |

#—p < 0.05 compared to baseline
$—p < 0.001 compared to baseline
NS—nonsignificant compared to baseline

As shown in above Table 2, there was significant improvement observed in endothelial function after 12 weeks of treatment with Shilajit (PrimaVie®) compared to baseline. With Shilajit (PrimaVie®) treatment there was significant reduction in augmentation index and significant increase in sub-endocardial ratio; whereas changes recorded in ABI, PWV and CO were not statistically significant compared to baseline values.

TABLE 2A
Comparison of Absolute change in Pharmacodynamic parameters
after 12 weeks of treatment with Shilajit (PrimaVie ®) &
Placebo - All values expressed as Mean ± SD

| Parameter | Shilajit (PrimaVie ®) | Placebo |
|---|---|---|
| RI (%) | −6.07 ± 2.81 $ | 2.08 ± 3.00 |
| AIx (%) | −8.59 ± 10.61 # | 0.69 ± 1.31 |
| SEVR(%) | 10.25 ± 13.76 # | 0.72 ± 2.29 |
| ABI | 0.01 ± 0.07 ns | 0.01 ± 0.01 |
| PWV(cm/s) | −82.5 ± 146 ns | 2.50 ± 33.44 |
| CO (Lt/min) | 0.2 ± 0.44 ns | −0.11 ± 0.24 |

$—RI - p < 0.001 Shilajit (PrimaVie ®) Vs Placebo
#—AIx - p < 0.05 Shilajit (PrimaVie ®) Vs Placebo
#—SEVR - p < 0.05 Shilajit (PrimaVie ®) Vs Placebo
ABI - Non-significant between the two treatments
PWV - Non-significant between the two treatments
CO - Non-significant between the two treatments

TABLE 3
Effect of Shilajit (PrimaVie ®) & Placebo on biomarkers after 12 weeks of
treatment - All values expressed as Mean ± SD

| | Shilajit (PrimaVie ®) n = 10 | | Placebo n = 10 | |
|---|---|---|---|---|
| Parameter | Pretreatment | Post treatment | Pretreatment | Post treatment |
| NO (μMol/L) | 29.40 ± 13.21 | 35.69 ± 13.75 # | 31.31 ± 8.20 | 30.81 ± 7.04 |
| MDA (nMol/ml) | 3.27 ± 0.78 | 2.65 ± 0.70 # | 3.22 ± 0.79 | 3.26 ± 0.72 |
| GSH (μMol/L) | 510.3 ± 120.4 | 639.4 ± 113.6* | 503.1 ± 47.29 | 502.7 ± 47.17 |
| HsCRP (mg/L) | 1.94 ± 0.89 | 0.87 ± 0.21 # | 2.11 ± 0.97 | 2.16 ± 0.96 |

*p < 0.05 compared to baseline
#—p < 0.01 compared to baseline

As shown in above Table 3, there were significant increases recorded in nitric oxide and glutathione levels in the Shilajit (PrimaVie®) treatment group compared to baseline. On treatment with Shilajit (PrimaVie®) there were also significant decreases in malondialdehyde and HsCRP levels observed compared to baseline.

TABLE 3A
Comparison of Absolute change in Biomarkers after 12 weeks of
treatment with Shilajit (PrimaVie ®) & Placebo -
All values expressed as Mean ± SD

| Parameter | Shilajit (PrimaVie ®) | Placebo |
|---|---|---|
| NO (μMol/L) | 6.30 ± 5.82 @ | −0.50 ± 2.98 |
| MDA (nMol/ml) | −0.63 ± 0.55 # | 0.05 ± 0.77 |
| GSH (μMol/L) | 129.09 ± 169.90 # | −0.34 ± 5.78 |
| HsCRP (mg/L) | −1.08 ± 0.89 $ | 0.05 ± 0.11 |

@—NO - p < 0.01 Shilajit (PrimaVie ®) Vs Placebo
#—MDA - p < 0.05 Shilajit (PrimaVie ®) Vs Placebo
#—GSH - p < 0.05 Shilajit (PrimaVie ®) Vs Placebo
$—HsCRP - p < 0.001 Shilajit (PrimaVie ®) Vs Placebo

TABLE 3B
Mean Percent change in Biomarkers after 12 weeks of treatment with
Shilajit (PrimaVie ®) & Placebo - All values expressed as Mean ± SD

| Parameter | Shilajit (PrimaVie ®) | Placebo |
|---|---|---|
| NO (%) | 24.26 ± 24.63 @ | −0.45 ± 10.45 |
| MDA (%) | −18.28 ± 16.77 | −0.35 ± 27.08 |
| GSH (%) | 33.02 ± 43.18 # | −0.07 ± 1.15 |
| HsCRP (%) | −44.71 ± 36.16 $ | 2.56 ± 6.73 |

@—NO - p < 0.01 Shilajit (PrimaVie ®) Vs Placebo
MDA - Non-significant Shilajit (PrimaVie ®) Vs Placebo
#—GSH - p < 0.05 Shilajit (PrimaVie ®) Vs Placebo
$—HsCRP - p < 0.001 Shilajit (PrimaVie ®) Vs Placebo

TABLE 4
Effect of Shilajit (PrimaVie ®) & Placebo after 12 weeks of treatment on lipid profile

| | Shilajit (PrimaVie ®) n = 10 | | Placebo n = 10 | |
|---|---|---|---|---|
| Parameter | Pretreatment | Post treatment | Pretreatment | Post |
| Total | 174.2 ± 26.68 | 139.4 ± 39.50 # | 173.2 ± 21.03 | 180.1 ± 18.65 |
| cholesterol (mg/dl) | | | | |
| HDL (mg/dl) | 39.20 ± 4.63 | 44.40 ± 6.81* | 40.60 ± 4.64 | 39.20 ± 4.59 |
| LDL (mg/dl) (mg/dl) | 105.5 ± 20.58 | 91.40 ± 18.06 # | 109.5 ± 24.19 | 112.0 ± 22.17 |
| Triglycerides (mg/dl) | 130.2 ± 42.08 | 104.7 ± 23.13 # | 145.0 ± 12.48 | 148.5 ± 15.46 |
| VLDL (mg/dl) | 29.40 ± 16.55 | 22.90 ± 8.64* | 31.00 ± 4.59 | 30.70 ± 4.85 |

*p < 0.05 compared to baseline
#—p < 0.01 compared to baseline

The above Table 4 indicates that, in the Shilajit (PrimaVie®) treatment group there were significant reductions in Total cholesterol, LDL-C, Triglycerides, and VLDL-C, compared to a significant increase in HDL-C levels compared to baseline.

TABLE 4A
Comparison of Absolute change in Lipid profile after 12 weeks
of treatment with Shilajit (PrimaVie ®) & Placebo -
All values expressed as Mean ± SD

| Parameter | Shilajit (PrimaVie ®) | Placebo n = 10 |
|---|---|---|
| Total cholesterol (mg/dl) | −34.80 ± 23.52 $ | 6.9 ± 12.28 |
| HDL (mg/dl) | 5.20 ± 6.92 # | −1.4 ± 2.59 |
| LDL (mg/dl) | −14.10 ± 11.33 $ | 2.5 ± 5.66 |
| Triglycerides (mg/dl) | −25.46 ± 24.30 @ | 3.5 ± 7.81 |
| VLDL (mg/dl) | −6.50 ± 8.80 # | −0.3 ± 2.00 |

$—Total cholesterol - p < 0.001 Shilajit (PrimaVie ®) Vs Placebo
#—HDL - p < 0.05 Shilajit (PrimaVie ®) Vs Placebo
$—LDL - p < 0.001 Shilajit (PrimaVie ®) Vs Placebo
@—Triglycerides - p < 0.01 Shilajit (PrimaVie ®) Vs Placebo
#—VLDL - p < 0.05 Shilajit (PrimaVie ®) Vs Placebo

TABLE 4B
Mean Percent change in Lipid Profile after 12 weeks of treatment
with Shilajit (PrimaVie ®) & Placebo -
All values expressed as Mean ± SD

| Parameter | Shilajit (PrimaVie ®) | Placebo n = 10 |
|---|---|---|
| Total cholesterol (%) | −20.67 ± 14.16 | 4.35 ± 6.93 |
| HDL (%) | 14.02 ± 19.51 | −3.24 ± 6.54 |
| LDL (%) | −12.90 ± 10.67 | 2.87 ± 5.44 |
| Triglycerides (%) | −16.19 ± 15.15 | 2.38 ± 5.24 |
| VLDL (%) | −16.52 ± 13.75 | −0.90 ± 6.45 |

TABLE 5
Effect of Shilajit (PrimaVie ®) & Placebo after 12 weeks of treatment on HbA1c (%)

| | Shilajit (PrimaVie ®) n = 10 | | Absolute | Mean percentage | Placebo n = 10 | | Absolute | Mean percentage |
|---|---|---|---|---|---|---|---|---|
| Parameter | Pre treatment | Post treatment | change | change | Pre treatment | Post treatment | change | change |
| HbA1c (%) | 7.73 ± 0.54 | 6.78 ± 0.43 $ | −0.95 ± 0.49 | −12.10 ± 5.63 | 7.48 ± 0.47 | 7.52 ± 0.51 | 0.04 ± 0.18 | 0.54 ± 2.35 |

$ -p < 0.001 compared to baseline
In Absolute change
p < 0.001 Shilajit (PrimaVie ®) Vs placebo

The above Table 5 shows that, in the Shilajit (PrimaVie®) treatment group there was a significant decrease in glycosylated hemoglobin A1c levels (HbA1c) observed compared to baseline. When a comparison between Shilajit (PrimaVie®) and placebo was performed there was statistical significance observed in absolute change.

TABLE 6
Effect of Shilajit (PrimaVie ®) and Placebo on Platelet Function-
Percentage decrease in inhibition of Platelet aggregation
(All values expressed as Mean ± SD)

| Group | Pretreatment | Post treatment | % Inhibition |
|---|---|---|---|
| Shilajit | 77.40 ± 11.64 | 66.50 ± 9.20 # $ | 13.73 ± 6.88 |
| (PrimaVie ®) | | | |
| n = 10 | | | |
| Placebo | 69.20 ± 5.26 | 70.10 ± 6.31 | 1.97 ± 2.89 |

#—p < 0.001compared to baseline
$—p < 0.001 Shilajit (PrimaVie ®) Vs Placebo
$\% Inhibition calculation = \frac{( Pre treatment Aggregation - Post treatment Aggregation )}{Pre treatment Aggregation} \times 100$

As shown in above Table 6, there was a significant decrease in platelet aggregation after treatment with Shilajit (PrimaVie®) compared to baseline. There was a statistically significant change in percentage decrease in platelet aggregation observed when a comparison was performed between Shilajit (PrimaVie®) and placebo.

TABLE 7
Effect of Shilajit (PrimaVie ®) and Placebo on safety parameters (all values expressed as Mean ± SD)

| | Shilajit (PrimaVie ®) n = 10 | | Placebo n = 10 | |
|---|---|---|---|---|
| Parameters | Pretreatment | Post treatment | Pretreatment | Post treatment |
| Systolic BP (mmHg) | 119.40 ± 4.01 | 118.20 ± 2.39 | 116.60 ± 3.13 | 117.20 ± 3.01 |
| Diastolic BP (mmHg) | 76.20 ± 5.37 | 77.00 ± 4.92 | 74.40 ± 4.09 | 75.20 ± 3.43 |
| Heart rare (bpm) | 77.40 ± 3.78 | 75.20 ± 4.02 | 74.40 ± 4.50 | 76.20 ± 3.19 |
| Hemoglobin (gm/dl) | 12.93 ± 1.18 | 13.43 ± 1.01 | 13.15 ± 1.23 | 14.05 ± 1.22 |
| WBC Count (/mm³) | 7190.00 ± 1198.56 | 6980.00 ± 784.29 | 6530.00 ± 1064.63 | 7010.00 ± 938.62 |
| Platelet Count (lakh/mm³) | 2.28 ± 0.66 | 2.65 ± 0.70 | 2.10 ± 0.80 | 2.34 ± 0.72 |
| Blood Urea (mg/dl) | 24.60 ± 8.38 | 26.60 ± 5.82 | 22.10 ± 7.17 | 25.10 ± 6.12 |
| S. Creatinine (mg/dl) | 0.98 ± 0.12 | 1.00 ± 0.11 | 1.03 ± 0.16 | 1.05 ± 0.16 |
| AST (SGOT) (U/L) | 19.60 ± 8.49 | 21.50 ± 8.67 | 24.20 ± 7.54 | 26.20 ± 7.07 |
| ALT (SGPT) (U/L) | 24.70 ± 6.50 | 25.30 ± 5.25 | 23.70 ± 6.40 | 26.20 ± 6.75 |
| Alkaline Phosphatase (U/L) | 184.60 ± 46.07 | 189.60 ± 29.35 | 163.00 ± 42.83 | 158.40 ± 38.06 |
| Total Bilirubin (mg/dl) | 0.54 ± 0.26 | 0.49 ± 0.18 | 0.52 ± 0.27 | 0.55 ± 0.16 |

As shown in above Table 7, at post treatment, there were no significant changes in hematological, renal and hepatic functions. There was no serious adverse event recorded in the study.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, water, fruit and/or vegetable juices, and combinations thereof.

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, a Shilajit composition may be further combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound(s). Suitable carriers are magnesium carbonate, magnesium state, talc, sugar, lactose, pectin, dextrin, starch, gelatin, tragacanth, methylcellulose, sodium carboxymethlycellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for administration in the mouth include lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearates, stearic acid, silicone fluid, talc, waxes, oils, colloidal silica, and the like. The usefulness of such excipients is well known in the art.

In one embodiment, the nutritional composition may be in the form of a liquid. In accordance with this embodiment, a method of making a liquid composition is provided.

Liquid nutritional compositions for oral administration in connection with a method for preventing and/or endothelial dysfunction or cardiovascular disorders including diabetes can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (RTD's) are contemplated.

Routes of Administration

The compositions may be administered by any suitable route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped artices, e.g. films or microcapsules.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. A method of treating or preventing endothelial dysfunction comprising administering to an individual in need thereof an effective amount of a composition comprising Shilajit and a pharmaceutically acceptable carrier, wherein endothelial function is improved.

2. The method according to claim 1 wherein the Shilajit includes at least about 50% by weight fulvic acids (FAs), at least about 10% by weight dibenzo-α-pyrone chromoproteins, and at least about 0.3% by weight total dibenzo-α-pyrones (DBPs) based on the total weight of the composition.

3. The method according to claim 2 wherein the Shilajit includes at least about 60% by weight fulvic acids (FAs) based on the total weight of the composition.

4. The method according to claim 2 wherein the improved endothelial function includes an increase of at least about 20% in the blood level of nitric oxide (NO).

5. The method according to claim 2 wherein the improved endothelial function includes an increase of at least about 8% in subendocardial viability ratio (SEVR).

6. The method according to claim 2 wherein the improved endothelial function includes a decrease of at least about 6% in augmentation index (AIx).

7. The method according to claim 2 wherein the composition is administered in a dose of about 250 mg/day to about 1000 mg/day.

8. A method of treating a diabetic individual suffering from type 2 diabetes mellitus comprising administering to an individual in need thereof an effective amount of a composition comprising Shilajit and a pharmaceutically acceptable carrier, wherein endothelial function is improved.

9. The method according to claim 8 wherein the Shilajit includes at least about 50% by weight fulvic acids (FAs), at least about 10% by weight dibenzo-α-pyrone chromoproteins, and at least about 0.3% by weight total dibenzo-α-pyrones (DBPs) based on the total weight of the composition.

10. The method according to claim 9 wherein the Shilajit includes at least about 60% by weight fulvic acids (FAs) based on the total weight of the composition.

11. The method according to claim 9 wherein the improved endothelial function includes an increase of at least about 20% in the blood level of nitric oxide (NO) in the diabetic individual.

12. The method according to claim 9 wherein the improved endothelial function includes an increase of at least about 25% in the blood level of nitric oxide (NO) in the diabetic individual.

13. The method according to claim 9 wherein the improved endothelial function includes an increase of at least about 30% in the blood level of high sensitivity C-reactive protein (HsCRP) in the diabetic individual.

14. The method according to claim 9 wherein the improved endothelial function includes an increase of at least about 25% in the blood level of glutathione (GSH) in the diabetic individual.

15. The method according to claim 9 wherein the improved endothelial function includes an increase of at least about 8% in subendocardial viability ratio (SEVR) in the diabetic individual.

16. The method according to claim 9 wherein the improved endothelial function includes a decrease of at least about 6% in augmentation index (AIx) in the diabetic individual.

17. The method according to claim 9 wherein the composition is administered in a dose of about 250 mg/day to about 1000 mg/day.

18. A method of treating a diabetic individual suffering from type 2 diabetes mellitus comprising administering to an individual in need thereof an effective amount of a composition comprising Shilajit and a pharmaceutically acceptable carrier, wherein a blood lipid parameter is improved.

19. The method according to claim 18 wherein the Shilajit includes at least about 50% by weight fulvic acids (FAs), at least about 10% by weight dibenzo-α-pyrone chromoproteins, and at least about 0.3% by weight total dibenzo-α-pyrones (DBPs) based on the total weight of the composition.

20. The method according to claim 19 wherein the Shilajit includes at least about 60% by weight fulvic acids (FAs) based on the total weight of the composition.

21. The method according to claim 19 wherein the improved blood lipid parameter includes a decrease of at least about 10% in the blood level of total cholesterol or LDL-C in the diabetic individual.

22. The method according to claim 19 wherein the improved blood lipid parameter includes a decrease of at least about 20% in the blood level of total cholesterol in the diabetic individual.

23. The method according to claim 19 wherein the improved blood lipid parameter includes a decrease of at least about 10% in the blood level of glycosylated haemoglobin percent (HbA1c %) in the diabetic individual.

24. The method according to claim 19 wherein the composition is administered in a dose of about 250 mg/day to about 1000 mg/day.
